(19) United States
(12) Patent Application Publication
(10) Pub. No.: US 2025/0241970 A1
(43) Pub. Date: Jul. 31, 2025

(54) TRADITIONAL CHINESE MEDICINE FOR TREATING HEMORRHOIDS

(71) Applicants: Qinghua Zhao, Taian (CN); Shicong Yang, Hangzhou (CN)

(72) Inventors: Qinghua Zhao, Taian (CN); Shicong Yang, Hangzhou (CN)

(21) Appl. No.: 18/603,817

(22) Filed: Mar. 13, 2024

(30) Foreign Application Priority Data
Jan. 29, 2024 (CN) 2024101167208

(51) Int. Cl. A61K 36/03; A61K 31/045; A61K 33/36; A61K 36/232; A61K 36/28; A61K 36/328; A61K 36/489; A61K 36/534; A61K 36/535; A61K 36/708; A61K 36/758; A61K 36/9066

(52) U.S. Cl. CPC A61K 36/03; A61K 31/045; A61K 33/36; A61K 36/232; A61K 36/28; A61K 36/328; A61K 36/489; A61K 36/534; A61K 36/535; A61K 36/708; A61K 36/758; A61K 36/9066

(57) ABSTRACT

A traditional Chinese medicine for treating hemorrhoids comprises the following raw materials: borneol, Centipeda minima, Pericarpium Zanthoxyli, Radix Sophorae Flavescentis, sargassum, Radix et Rhizoma Rhei, Radix Curcumae, Radix Angelicae Dahuricae, Folium Perillae, Herba Menthae Haplocalycis, processed myrrha, realgar. The formulation design of the invention is based on a rigorous and rational analysis of the pathogenesis, pathology, and meridian tropism logic. The treatment of hemorrhoids can achieve the effects of relieving pain and stopping bleeding, softening the hardness and eliminating the nodules, eliminating putrefaction and promoting granulation, and treating both symptoms and root causes. The invention is quick to relieve pain and stop bleeding, has a short healing time, can treat hemorrhoids in different locations such as internal hemorrhoids, external hemorrhoids, and mixed hemorrhoids, and can also treat constipation, rectal prolapse, anal abscess, rectal cancer, perianal eczema and other related anorectal diseases.

### 1. TECHNICAL FIELD

The invention relates to the technical field of traditional Chinese medicine, in particular to a traditional Chinese medicine for treating hemorrhoids.

### 2. BACKGROUND ART

Hemorrhoids, also known as piles, are soft vein masses formed by varicosity of submucosal veins around the end of the rectum and subcutanceous veins of the anal canal and the anal margin; or skin inflammation, hypertrophy, and connective tissue hyperplasia of the folds around the anal margin; or anal fissures, or lumps, sores and ulcers formed by the syndrome of dampness-heat diffused downward, qi stagnation and blood stasis, pathogenic dampness and heat toxin.

Hemorrhoids are a unique disease in humans. They can occur at any age, but clinically they are most common in those between 20 and 40 years old. As age increases, the incidence gradually rises. There is a saying that goes “nine out of ten men have hemorrhoids” and “ten out of ten women have hemorrhoids”. According to anatomical location and symptom presentation, hemorrhoids are classified as internal hemorrhoids, external hemorrhoids, or mixed hemorrhoids. The modern medical treatment principle for hemorrhoids mainly focuses on surgery to remove the hemorrhoids to eliminate symptoms. However, surgery is expensive, has a high recurrence rate, and can damage the physiological structure around the anus, leading to postoperative complications such as perianal stenosis and anal fissures. In China, most medications used are external OTC drugs, such as Rongchang Gangtai and Mayinglong Hemorrhoid Ointment. These medications only provide relief for mild hemorrhoid symptoms, but the effectiveness is not significant for moderate to severe patients, with a very low cure rate. The pathogenesis and etiology of hemorrhoids are mainly caused by the invasion of the four evils: wind, dryness, dampness, and heat. Factors such as alcohol consumption, excessive intake of spicy foods, long-term constipation, diarrhea, prolonged sitting or standing, heavy lifting, pregnancy, and other factors can lead to the internal generation of dryness-heat or damp-heat, which presses down on the large intestine, blocks the meridians, and results in the accumulation of pathogenic heat and stagnant blood. The onset of hemorrhoids often causes unbearable suffering for patients, with symptoms such as hematochezia, pain, itching, and so on. Severe hemorrhoids can also lead to complications such as rectal cancer, perianal eczema, anemia, anal fissures, anal fistulas, perianal abscesses, prolapse of hemorrhoids, incarceration, and even tissue necrosis, seriously affecting the patient's life and quality of life.

Therefore, a traditional Chinese medicine for treating hemorrhoids has become an urgent problem for people to solve.

### 3. SUMMARY OF THE INVENTION

The object of the invention is to provide an efficient and convenient traditional Chinese medicine for treating hemorrhoids, which can effectively address the medication issues of hemorrhoids and anorectal related diseases.

To realize the above object, the invention provides the following technical scheme: a traditional Chinese medicine for treating hemorrhoids comprises the following raw materials in parts by weight: 40˜60 parts of borneol, 20˜40 parts of Centipeda minima, 20˜40 parts of Pericarpium Zanthoxyli, 8˜18 parts of Radix Sophorae Flavescentis, 7˜20 parts of sargassum, 6˜15 parts of Radix et Rhizoma Rhei, 5˜14 parts of Radix Curcumae, 5˜15 parts of Radix Angelicae Dahuricae, 5˜13 parts of Folium Perillae, 3˜12 parts of Herba Menthae Haplocalycis, 7˜20 parts of processed myrrha, 6˜16 parts of realgar.

Further, it comprises the following raw materials in parts by weight: 40 parts of borneol, 40 parts of Centipeda minima, 30 parts of Pericarpium Zanthoxyli, 16 parts of Radix Sophorae Flavescentis, 7 parts of sargassum, 12 parts of Radix et Rhizoma Rhei, 14 parts of Radix Curcumae, 5 parts of Radix Angelicae Dahuricae, 10 parts of Folium Perillae, 8 parts of Herba Menthae Haplocalycis, 15 parts of processed myrrha, 6 parts of realgar.

Compared with existing technologies, the advantages of the invention are as follows: the formulation design of the invention is based on a rigorous and rational analysis of the pathogenesis, pathology, and meridian tropism logic. The treatment of hemorrhoids can achieve the effects of relieving pain and stopping bleeding, softening the hardness and eliminating the nodules, eliminating putrefaction and promoting granulation, and treating both symptoms and root causes. The invention is quick to relieve pain and stop bleeding, has a short healing time, and can treat constipation, rectal prolapse, anal abscess, rectal cancer, perianal eczema and other related anorectal diseases.

The invention effectively addresses various symptoms of hemorrhoids and achieves a therapeutic effect by treating both symptoms and root causes; it is quick to relieve pain and stop bleeding, has a short healing time, and can treat a variety of anorectal diseases.

### 4. SPECIFIC EMBODIMENT OF THE INVENTION

The traditional Chinese medicine for treating hemorrhoids of the invention will be further described in detail below.

The formula of the invention:
borneol: pungent, bitter in taste, and slightly cold in nature; the medicinal effect acts on the heart, spleen, and lungs; it is used to treat coma due to blockage syndrome, red and swollen eyes, aphthous sores, pharyngitis, swelling and pain caused by sores and ulcers, sores and ulcers that do not heal after ulceration.
Centipeda minima: pungent in taste, warm in nature and non-toxic in property. It can unblock stuffy orifice and dispel cold, dispel wind and dampness, dissipate blood stasis and reduce swelling. Indications: colds, acute and chronic rhinitis, chronic bronchitis, malaria, bruises, rheumatic arthralgia, ascaris intestinal obstruction, and poisonous snake bites.
Pericarpium Zanthoxyli: pungent in taste and warm in nature; the medicinal effect acts on the stomach, spleen and kidneys; warming the spleen and stomach and relieving pain, insecticidal, and antipruritic; it is used for cold pain in stomach duct, vomiting and diarrhea, and abdominal pain due to parasitic infestation, ascariasis; external use: eczema and itching.
Radix Sophorae Flavescentis: bitter in taste, cold in nature; the medicinal effect acts on the small intestine, liver, kidneys, and large intestine; clearing away heat and dampness, dispeling wind and killing parasites. Indications: damp-heat diarrhea, hematochezia with syndrome of intestinal wind injuring collaterals, jaundice, dysuria, edema, leukorrhagia, vulva pruritus, scabies, leprosy, skin itching, damp toxin sores.
Sargassum: cold in nature, bitter and salty in taste; the medicinal effect acts on the stomach, liver, and kidneys; softening the hardness and eliminating the nodules, removing phlegm, and diuresis. It is used for galls, scrofula, testicular swelling and pain, edema caused by phlegm-fluid retention.
Radix et Rhizoma Rhei: cold in nature and bitter in taste; the medicinal effect acts on the stomach, liver, and large intestine; purging sthenic heat and breaking stagnation, removing blood stasis. Indications: constipation caused by sthenic heat, dyspepsia, abdominal pain, acute appendicitis, acute infectious hepatitis, amenorrhea caused by blood stasis, toothache, epistaxis, acute conjunctivitis; external use: burns, purulent skin diseases, carbuncle and ulcers.
Radix Curcumae: pungent, bitter in taste, and cool in nature; the medicinal effect acts on the heart, gallbladder and liver; promoting qi circulation and removing blood stasis, calming the heart and relieving depression, promoting bile secretion and treating jaundice. It is used for amenorrhea, dysmenorrhea, distension and pain, tingling in chest and abdomen, febrile coma, epilepsy and madness, jaundice with red urine.
Radix Angelicae Dahuricae: pungent in taste and warm in nature; the medicinal effect acts on the stomach, lungs, and spleen; dispersing wind and removing dampness, removing obstruction in channels and relieving pain, reducing swelling and discharging pus. It is used for colds and headaches, eyebrow bone pain, nasal congestion, nasosinusitis, toothache, leucorrhea, swelling and pain caused by carbuncle and sores.
Folium Perillae: pungent in taste and warm in nature; the medicinal effect acts on the lungs and spleen; relieving superficies syndrome by means of diaphoresis, dispelling cold, regulating qi, and harmonizing nutrient-blood. It can treat cold (wind-cold syndrome), cold with fever, cough, asthma, chest and abdominal distension, and fetal irritability. It can also resolve the fish and crab poisoning.
Herba Menthae Haplocalycis: pungent in taste and cool in nature; the medicinal effect acts on lungs and liver; dispersing wind, dissipating heat, expelling toxins from the body, and detoxifying. It can treat exogenous wind-heat, headache, red eyes, sore throat, indigestion and stomach bloating, aphtha, toothache, scabies, and urticaria.
Processed myrrha: bitter, pungent in taste, and flat in nature; the medicinal effect acts on the liver. It has the functions of promoting blood circulation and relieving pain, reducing swelling and promoting granulation. It can be used to treat bruises, swelling and pain caused by blood stasis, stomach pain caused by blood stasis, amenorrhea, dysmenorrhea, swelling and pain due to carbuncle, sores and ulcers that do not heal after ulceration.
Realgar: pungent in taste and warm in nature; the medicinal effect acts on the liver and large intestine. It can detoxify and kill parasites, eliminate dampness and phlegm, prevent attack of malaria and relieve convulsions. It not only cures sores, scabies, snake bites and abdominal pain due to parasitic infestation, but also cures asthma, malaria and epilepsy.

The invention is introduced in detail below.

A traditional Chinese medicine for treating hemorrhoids comprises the following raw materials in parts by weight: 40˜60 parts of borneol, 20˜40 parts of Centipeda minima, 20˜40 parts of Pericarpium Zanthoxyli, 8˜18 parts of Radix Sophorae Flavescentis, 7˜20 parts of sargassum, 6˜15 parts of Radix et Rhizoma Rhei, 5˜14 parts of Radix Curcumae, 5˜15 parts of Radix Angelicae Dahuricae, 5˜13 parts of Folium Perillae, 3˜12 parts of Herba Menthae Haplocalycis, 7˜20 parts of processed myrrha, 6˜16 parts of realgar.

The specific implementation process of a traditional Chinese medicine for treating hemorrhoids of the invention is as follow:
taking 40 parts of borneol, 40 parts of Centipeda minima, 30 parts of Pericarpium Zanthoxyli, 16 parts of Radix Sophorae Flavescentis, 7 parts of sargassum, 12 parts of Radix et Rhizoma Rhei, 14 parts of Radix Curcumae, 5 parts of Radix Angelicae Dahuricae, 10 parts of Folium Perillae, 8 parts of Herba Menthae Haplocalycis, 15 parts of processed myrrha, 6 parts of realgar;
drying at high temperature, disinfecting and sterilizing; then grinding it into fine powder according to the above proportion, about 170 mesh; making it into a suppository or powder spray or capsule, and inserting it into the anal sphincter muscle 2-3 cm.

The product made by the invention is quick to relieve pain and stop bleeding, has a short healing time, can treat both symptoms and root causes and can treat a variety of anorectal diseases.

The invention and its embodiments are described above, this description is not restrictive. If those of ordinary skill in the art receive its enlightenment, without deviating from the spirit of the invention, can design structural methods and embodiments similar to the technical scheme of the invention without creativity, which shall fall within the protection scope of the invention.

1. A traditional Chinese medicine for treating hemorrhoids comprises the following raw materials in parts by weight: 40˜60 parts of borneol, 20˜40 parts of Centipeda minima, 20˜40 parts of Pericarpium Zanthoxyli, 8˜18 parts of Radix Sophorae Flavescentis, 7˜20 parts of sargassum, 6˜15 parts of Radix et Rhizoma Rhei, 5˜14 parts of Radix Curcumae, 5˜15 parts of Radix Angelicae Dahuricae, 5˜13 parts of Folium Perillae, 3˜12 parts of Herba Menthae Haplocalycis, 7˜20 parts of processed myrrha, 6˜16 parts of realgar.

2. A traditional Chinese medicine for treating hemorrhoids of claim 1, it comprises the following raw materials in parts by weight: 40 parts of borneol, 40 parts of Centipeda minima, 30 parts of Pericarpium Zanthoxyli, 16 parts of Radix Sophorae Flavescentis, 7 parts of sargassum, 12 parts of Radix et Rhizoma Rhei, 14 parts of Radix Curcumae, 5 parts of Radix Angelicae Dahuricae, 10 parts of Folium Perillae, 8 parts of Herba Menthae Haplocalycis, 15 parts of processed myrrha, 6 parts of realgar.
